(19) United States
(12) Patent Application Publication
(10) Pub. No.: US 2016/0024558 A1
(43) Pub. Date: Jan. 28, 2016

(54) NUCLEIC ACID BINDING PROTEINS AND USES THEREOF

(71) Applicant: 10X Genomics, Inc., Pleasanton, CA (US)

(72) Inventors: Paul Hardenbol, San Francisco, CA (US); Keith Bjornson, Fremont, CA (US)

(21) Appl. No.: 14/806,471

(22) Filed: Jul. 22, 2015

Related U.S. Application Data
(60) Provisional application No. 62/027,974, filed on Jul. 23, 2014.

(51) Int. Cl. C12Q 1/68

(52) U.S. Cl. CPC C12Q 1/6806

(57) ABSTRACT

Compositions, systems and methods employing nucleic acid binding proteins for use in the regulation and/or modulation of nucleic acid based reactions, including transcription, translation, modification, digestion, and hybridization reactions. Such compositions are employed in controlling a variety of different reaction types involving nucleic acids.

### CROSS-REFERENCE TO RELATED APPLICATIONS

This application claims priority to U.S. Provisional Patent Application No. 62/027,974, filed Jul. 23, 2014, the full disclosure of which is herein incorporated by reference in its entirety for all purposes.

### BACKGROUND

The field of life sciences has experienced dramatic advancement over the last two decades. From the broad commercialization of products that derive from recombinant DNA technology, to the simplification of research, development and diagnostics, enabled by the invention and deployment of critical research tools, such as the polymerase chain reaction, nucleic acid array technologies, robust nucleic acid sequencing technologies, and more recently, the development and commercialization of high throughput next generation sequencing technologies. All of these improvements have combined to advance the fields of biological research, medicine, diagnostics, agricultural biotechnology, and a myriad of other related fields.

Intrinsic in the above analyses are wide ranging nucleic acid reactions, analyses, manipulations, and the like. It can be desirable to be able to modulate these reactions in a variety of ways for a variety of different applications. The present disclosure addresses these and other needs.

### SUMMARY

The present disclosure provides devices, systems and methods employing nucleic acid binding proteins.

An aspect of the disclosure provides a method of controlling a reaction with a nucleic acid. The method includes providing a nucleic acid comprising at least one uracil containing nucleotide and providing a nucleic acid binding protein with specificity for binding uracil containing nucleic acids in contact with the nucleic acid. The nucleic acid binding protein can be provided under conditions whereby the nucleic acid binding protein binds to a portion of the nucleic acid comprising the uracil containing nucleotide, whereby binding of the nucleic acid protein to the nucleic acid at least partially blocks a reaction with the nucleic acid.

In some cases, the reaction comprises a ligation reaction, a polymerization reaction, an exonuclease reaction, an endonuclease reaction, a protection reaction, and/or a hybridization reaction. In some cases, the reaction comprises a polymerization reaction. In some cases, the nucleic acid binding protein comprises an archeal polymerase, a uracil-DNA glycosylase, a uracil binding fragment or a construct thereof.

In some cases, the nucleic acid binding protein comprises an archeal polymerase or a uracil binding fragment or a construct thereof. In some cases, the reaction comprises a hybridization reaction and a reagent in the reaction comprises a polynucleotide complementary to at least a portion of the nucleic acid bound by the nucleic acid binding protein. In some cases, the reaction comprises a protection reaction, and a reagent in the reaction comprises an exonuclease or an endonuclease, and the nucleic acid binding protein reduces activity of such endonuclease or exonuclease on at least a portion of the nucleic acid bound by the nucleic acid binding protein.

An additional aspect of the disclosure provides a composition. The composition can include a nucleic acid comprising one or more uracil containing bases; a nucleic acid binding protein with specificity for binding uracil containing nucleic acids, bound to the nucleic acid; and a reagent capable of reacting with the nucleic acid. The presence of a bound nucleic acid binding protein can at least partially block the reagent from reacting with the nucleic acid.

In some cases, the reagent comprises an enzyme that can be a polymerase, a ligase, a transcriptase, an endonuclease or an exonuclease. In some cases, the reagent comprises a polymerase. In some cases, the nucleic acid binding protein comprises an archeal polymerase, a uracil-DNA-glycosylase, a uracil binding fragments or a construct thereof. In some cases, the nucleic acid binding protein comprises an archeal polymerase or a uracil binding fragment or construct thereof. In some cases, the archeal polymerase comprises 9° North polymerase, a uracil binding fragment or a construct thereof. In some cases, the 9° North polymerase comprises a polymerase inactive construct of 9° North polymerase. In some cases, the 9° North polymerase comprises an exonuclease deficient 9° North polymerase.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

FIG. 1 schematically illustrates an example of a nucleic acid binding protein for use in controlling or modulating nucleic acid based reactions, such as nucleic acid replication reactions.

FIG. 2 demonstrates high affinity binding of nucleic acid binding proteins to targeted nucleic acids.

FIG. 3 illustrates the concentration dependent inhibition of polymerase activity through the use of nucleic acid binding proteins.

FIG. 4 illustrates blockading of strand displacing polymerase activity using nucleic acid binding proteins as described herein.

### DETAILED DESCRIPTION

A wide variety of biological operations, analyses, and manipulations employ nucleic acid reactions as at least one component. These include, among many different operations, genetic recombination to produce new proteins and polypeptides, up and down-regulation of genetic components to impact the overall biochemical operation of an organism or components thereof, cloning of genetic components from one organism to another, and analysis of the genetic makeup of organisms or their constituent parts.

For a variety of these operations, the ability to more precisely control the participation of nucleic acids in reactions would be highly desirable. For example, where introducing genetic components into a biological system one may desire to control when and if the genetic component is subject to expression and transcription. Likewise, one may desire the ability to control the initiation of replication reactions in different analytical reactions, e.g., rtPCR, DNA sequencing, or the like, in order to ensure a simultaneous or near simultaneous start of replication for nucleic acid molecules within a mixture, often termed a “hot start”.

The present disclosure is directed to methods, compositions and systems useful in controlling nucleic acid based reactions. In particular, provided are nucleic acids that include one or more nucleotides that operate as affinity binding loci for one or more nucleic acid binding proteins put into contact with those nucleic acids. The affinity binding of the nucleic acid binding proteins with the nucleic acids impacts, and in many cases, substantially inhibits interaction of the nucleic acid with other reagents that would normally be capable of reacting with that nucleic acid in the absence of the binding protein, including, e.g., hybridization based interactions (e.g., primer template associations, capture reactions, splinted ligation reactions, and the like), nucleic acid processing reactions (e.g., replication, transcription or translation reactions, amplification reactions, and the like).

In one aspect, the nucleic acid binding proteins provided herein include those that recognize and bind to specific nucleotides or nucleotide sequences in polynucleotide sequences. These specific nucleotides or nucleotide sequences may be included within target segments which are exposed to the nucleic acid binding proteins in order to use that binding as a modulating influence on the reaction of those nucleic acids with other reactants.

In a first example, the nucleic acid binding proteins include, e.g., binding proteins or protein components that bind to uracil containing bases such as in nucleic acid sequences, including oligo and polynucleotide sequences, also referred to herein as U-binding proteins. In particular, there are a number of proteins that specifically associate with uracil containing bases, and in many cases, uracil containing deoxynucleotide bases in polynucleotide molecules. U-Binding proteins described herein may have relatively high affinities for uracil base containing nucleic acid sequences in the reaction conditions being exploited. In particular, such affinities may be represented by Ki that is in the low nanomolar range for the given construct and relevant reaction conditions. In general, such low nanomolar affinities may be less than 20 nM, less than 15 nM, less than 10 nM, less than 9 nM, less than 8 nM, less than 7 nM, less than 6 nM, less than 5 nM, less than 4 nM, less than 3 nM, less than 2 nM, or less than 1 nM, or between 1 nM and each of the other affinities specifically mentioned above, for the particular reaction conditions in which the U-binding protein is being exploited.

Examples of U-binding proteins include uracil binding DNA polymerases, such as the 9° North polymerases and polymerases from related archea, such as the Vent and Deep Vent polymerases, available from New England Biolabs, Inc. (both exo+ and exo− enzymes), VeraSeq polymerases from, e.g., Enzymatics, Inc., and pfu Polymerases, available from, e.g., Agilent. In general, the aforementioned polymerases have measured affinities for uracil containing nucleic acids in the low nanomolar range for relevant conditions. In some examples, the U-binding proteins may be employed both for their U-binding capabilities, as well as for their other enzymatic activities. For example, as described in greater detail below, in some instances, polymerases with U-binding activity may also be provided to carry out polymerization reactions with the associated or other nucleic acids within a reaction mixture. Alternatively or additionally, additional enzymes may be included for carrying out the desired reaction while using the U-binding protein for its U-binding activities instead of or in addition to its other enzyme activities. For example, in some cases, U-binding proteins, including polymerases (active, deactivated, or U-binding fragments thereof) may be included in addition to a non-U-binding polymerase, ligases, or the like, which are able to process the nucleic acid without being complexed by the U-containing nucleic acids. Moreover, in some cases, the U-binding polymerases, may be in forms or constructs that are active or inactive for polymerase activity, and may also retain or have reduced or eliminated other inherent activities, such as exonuclease activities. By way of example, in some cases an archeal polymerase may be employed as a U-binding protein, that has been constructed to remove its exonuclease and/or its polymerase activity. Polymerase inactive archeal polymerases have been described in e.g., Rogozin et al., Biol. Direct 2008, 3:32. Likewise, exonuclease deficient (e.g., inactive [exo−] or reduced activity [exo-down]) forms of such polymerases are available, e.g., in U.S. Pat. No. 5,756,334, which is incorporated herein by reference in its entirety for all purposes.

Similarly, other U-binding proteins may also be employed in this regard, including, e.g., uracil-DNA glycosylases (UDG) and related U-binding proteins or U-binding fragments or constructs thereof. U-binding proteins may include the full-length U-binding proteins described above, or any derivatives of those proteins that retain the U-binding activity. For example, polypeptides that retain the U-binding motif of any of the above-described polypeptides, e.g., the 199-GVLLLN-204 uracil binding motif in UDG, may be included. Similarly, for polymerases or other enzymes, the proteins in which the enzymatic activity has been knocked out or deactivated, in part or in whole, e.g., by mutation, cleavage or other processing, may also be included.

U-binding proteins can have a binding affinity for the uracil containing base that is strong enough to functionally inhibit the reaction that is desired to be controlled. For example, where one is using a U-binding protein to inhibit replication by a given DNA polymerase, it will be understood that the dissociation of the U-binding protein can inhibit any displacement activity of the polymerase, e.g., the U-binding affinity of the U-binding protein may inhibit the replication of the uracil containing nucleic acid region, e.g., by at least 10%, at least 20%, at least 30%, at least 40%, at least 50%, at least 60%, at least 70%, at least 80%, at least 90%, at least 100%, at least 200% or more, as compared to such replication in the absence of such U-binding proteins.

In operation, controlling reactions of a nucleic acid may include incorporating within the nucleic acid a uracil containing base. This may be included at any of a variety of desired locations, depending upon where the control is desired. For example, if it is desired to only replicate a portion of a sequence, a uracil containing base or bases may be inserted into the sequence just downstream of the portion of a nucleic acid that is desired to be replicated. Once that portion is bound to the U-binding protein, the replication of the nucleic acid would not proceed beyond the portion complexed with the U-binding protein. Accordingly, in some cases, compositions are provided that include a uracil containing nucleic acid and a U-binding protein as described herein. These compositions may include, or may have introduced to them, one or more reagents that act upon or react with the oligonucleotide in a manner that would be blocked by the U-binding protein. Examples of such action include enzymatic action or reaction, e.g., polymerases, ligases, endonucleases, exonucleases, where the activity of such enzyme may be sterically hindered by the presence of a proximal U-binding protein to reduce, inhibit or block such activity. As used herein, such reduction, inhibition or blocking may generally be characterized as being a reduction in the activity of the enzyme relative to such activity in the absence of the nucleic acid binding protein of at least about 50%, at least about 60%, at least about 70%, at least about 80%, at least about 90%, and in some cases at least about 95% or even at least about 99%.

In some cases, the added reagents may include the particular enzyme, as well as any ancillary reagents for the given reaction, e.g., nucleoside triphosphates, magnesium or manganese salts, and the like. In some cases, one may use the U-binding proteins described herein in protection processes, e.g., to remove extraneous nucleic acids from a mixture, e.g., through exo or endonuclease treatment of the resultant mixture, e.g., where the U-binding proteins are bound to the nucleic acid. In such cases, one may desire to position the U-binding protein at or near a given end of the nucleic acid to block an undesired level of exonuclease activity.

As will be appreciated, uracil containing bases may be included at any desired location in the nucleic acid of interest in order to achieve the desired results, and may be included at a single location, or at multiple locations. Likewise, one may add saturating amounts of U-binding proteins to completely bind the uracil bases in a given nucleic acid, or one may titrate different amounts of U-binding proteins to more controllably regulate an overall reaction, e.g., block some but not all replication or other reactions.

As noted above, although described in terms of regulating or modulating replication and other enzymatic reactions, it will be appreciated that a variety of different nucleic acid reactions may be modulated using these U-binding proteins, including, e.g., hybridizations reactions, transcription, translation, modification reactions, and the like. In such cases, the added reagents may include complementary polynucleotides, transcription effectors, e.g., reverse transcriptases, modification reagents, and the like. Likewise, these methods may be applied in a variety of applications, including amplification reactions, both for analysis and cloning, targeted assays, targeted pull-down reactions, and any other nucleic acid reactions of interest in which U-binding proteins may be used to modulate some or all of the reactions. In some cases, enrichment of nucleic acids may be enhanced, or reduced through the use of U-binding proteins to block binding of certain regions from hybridization with an enrichment probe set. For example, a U-binding protein may be employed to shield U-containing primers (and their direct extension products) from enrichment when using probes targeted for the non-uracil containing sequence segment for such primers, e.g., the non-uracil containing replicate of the original uracil containing sequence.

Alternatively, the U-binding protein may, itself, provide a specific binding target for enriching the uracil containing nucleic acids, e.g., through the use of a ligand specific for the U-binding protein, e.g., an antibody.

In some cases, it may be desirable to reverse the association of the U-binding protein from the nucleic acid. In general, this may be achieved through a number of processes, including, for example, purification of the proteins away from the nucleic acids, denaturation of the proteins, e.g., thermally or chemically, digestion of the proteins, e.g., using proteases, or the like. In such cases, the reactivity of the nucleic acid within a given system may be restored.

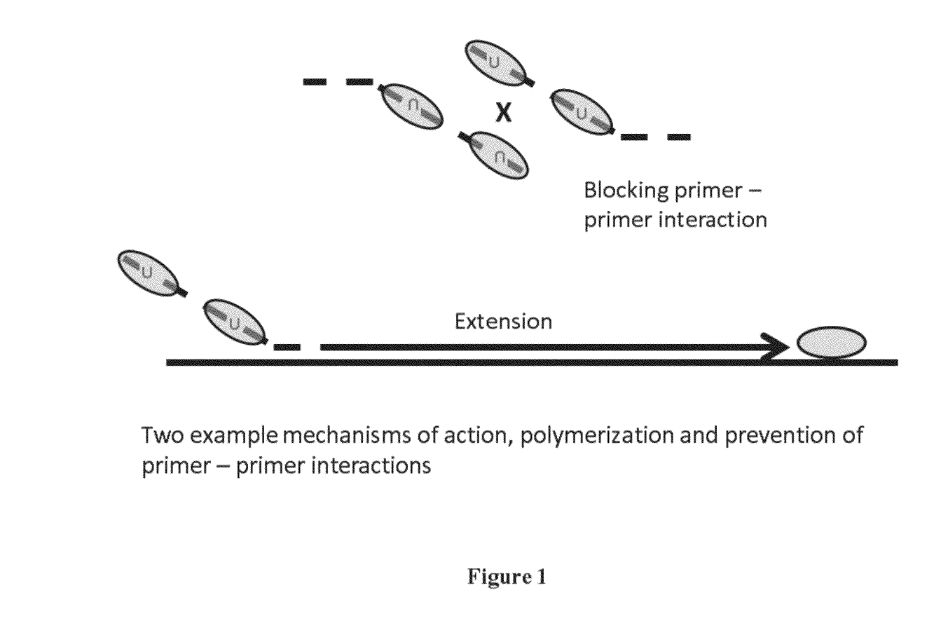

FIG. 1 schematically illustrates use of a U-binding protein, e.g., 9° North polymerase, as a blocking group on a portion of a nucleic acid that includes uracil containing bases, in order to prevent complete replication of the nucleic acid. This application can be useful in the replication of nucleic acids or partial replication of nucleic acids to provide useful structures, e.g., to create partial hairpin amplicons. Partial hairpin amplicons have a variety of valuable characteristics, including for example, their ability to self sequester from subsequent replication reactions, e.g., as used in preparation of sequencing libraries, as described in U.S. patent application Ser. No. 14/316,383, filed Jun. 26, 2014, the full disclosure of which is herein incorporated by reference in its entirety for all purposes. In some cases, by including uracil containing bases in a portion of the primer sequence (dashed), one can avoid copying of that primer section into any subsequent replication of the first copy. One clear benefit of such structures is in preventing the creation of primer complements that could hybridize with your original primer library, leading to the production of increased numbers of primer artifacts, e.g., primer-dimers, that decrease the quality of an amplified sample, e.g., for use in a sequencing library.

Additionally, one may employ such partial replication techniques to allow for control of replication of the original amplicon, e.g., by removing the blocking protein and subsequently allowing separate priming off the unreplicated or blocked region using a more permissive polymerase, e.g., a phi29, poll, or other polymerase, one can ensure only replication of an original amplicon.

### EXAMPLES

To analyze the nucleic acid binding characteristics of certain proteins, varying concentrations of an archeal DNA polymerase, 9° North Polymerase (obtained from New England Biolabs, Inc.) were incubated with uracil containing DNA oligonucleotide sequence in a gel shift assay as described by Shuttleworth, et al., J. Mol. Biol. (2004) 337, 621-634 (incorporated herein by reference in its entirety for all purposes). The gel shift analysis used 0.5 nM concentrations of a single-stranded 74-mer, and including 10 uracil nucleotides in one instance and without uracil in other. The 9° North polymerase (concentrations between 0 nM and 100 nM) was mixed with the oligodeoxynucleotide in volumes of 200 μl containing: 20 mM (pH 8.8), 10 mM KCl, 10 mM (NH₄)₂SO₄, 1 mM EDTA, 20 mg of bovine serum albumin and 0.1% (v/v) Triton-X100, and incubated at 72° C. for 20 minutes. Free and bound DNA bands were then separated on a native 10% (w/v) polyacrylarnide gel, and stained with SYBR Gold, and imaged using a standard gel imaging system.

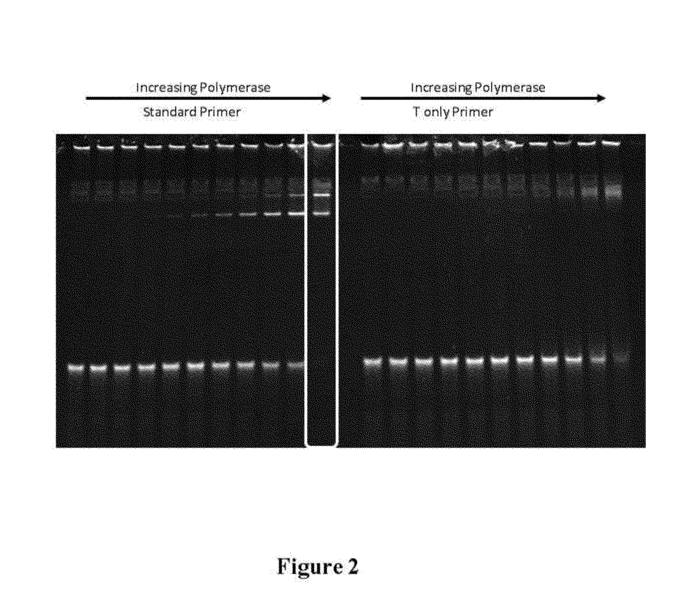

FIG. 2 presents the gels that show in the left panel, the binding of a labeled U-containing primer sequence (low molecular weight band), to increasing concentrations of 9° North polymerase (middle high molecular weight band). Further, the gel shows that the 9° North polymerase binds to at least two separate locations on the primer, as shown by the highest molecular weight band (representing two proteins bound to a single labeled primer). By comparison, the gel shown in the right side panel repeats the experiment with the same primer sequence, except where thymine containing bases are included in place of uracil containing bases. Moreover, sequence analysis of the amplicons produced in the presence of this U-binding activity resulted in amplicons that lack replicates of the U-containing portion, as blocked by the U-binding activity.

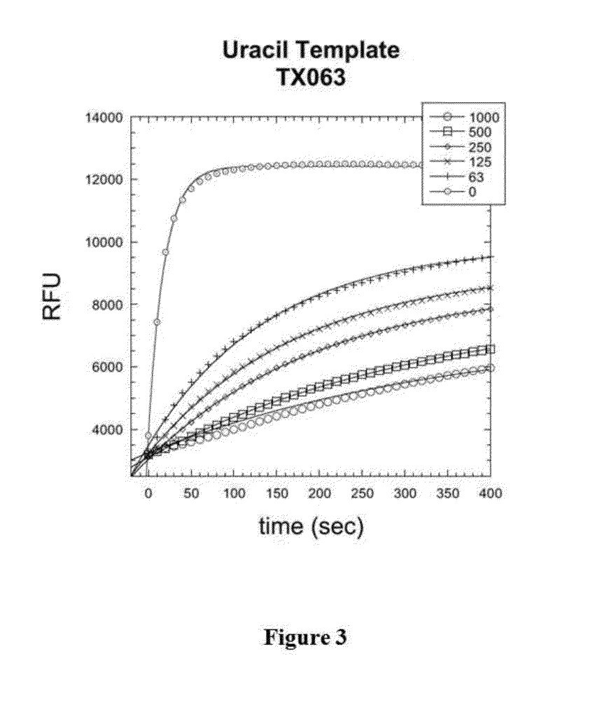

In a further experiment, 100 nM of primer template oligonucleotide /56-FAM/TCGAGCACGCGGCACTTATTGCAA/dideoxyU/AGTGCCGAGTCAGCGCGCTGACTC GGC annealed with 100 nM GCAATAAGTGCCGCGTGCTCGA/3IABkFQ/ to form a quenched duplex. The DNA duplex was incubated with the indicated nanomolar concentration of the polymerase inactive construct (Y538H, D540I, T541Y) (See, e.g., Rogozin et al., Biol. Direct 2008, 3:32) and 120 nM of active 9° North polymerase. The reaction was initiated with addition of 200 μM (dATP, dGTP, dCTP, and dTTP each) in the Reaction Buffer. The reaction was monitored using a BioRad CFX96 Deep Well real time PCR machine at 55° C. FIG. 3 illustrates the fluorescence enhancement observed upon polymerase activity that displaces the annealed oligonucleotide with the attached “Iowa Black” quencher. As shown, uracil dependent nucleic acid binding is shown by inhibition of duplex displacement on a concentration dependent basis for the inactive 9° North.

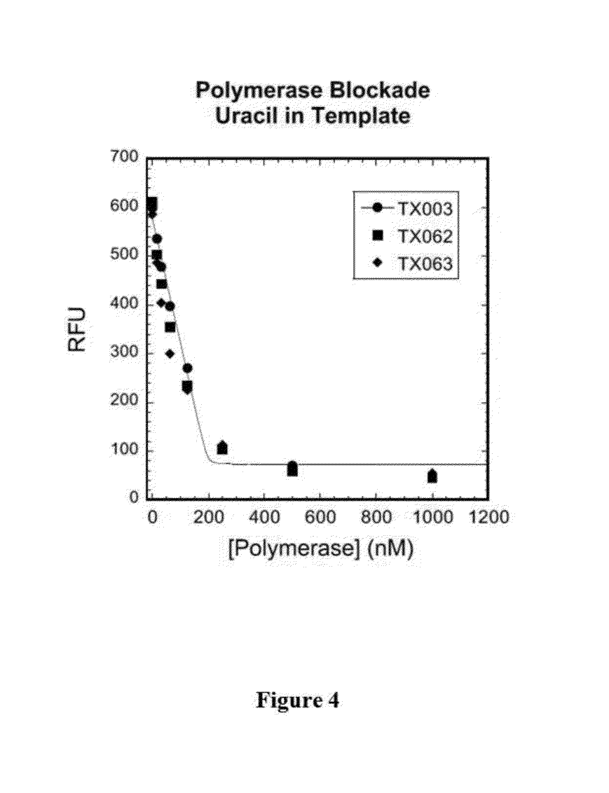

Next, the nucleic acid binding protein was examined for the ability to inhibit polymerase activity through the bound region of the oligonucleotide. Again, 100 nM of primer template /56-FAM/TCGAGCACGCGGCACTTATTGCAA/dideoxyU/AGGAAATTACCCTTTATGCGTGCCGAGTCAGCGCGCTGACTCGGC was annealed with 100 nM GCAATAAGTGCCGCGTGCTCGA/3IABkFQ/ to form a quenched duplex. The duplex was then incubated with both polymerase active (TX03, exo-down) and inactive (TX062 and TX063 exo-down) forms of the 9° North polymerase at varied concentrations, along with 50 mM Tris-HCl pH 7.5, 10mM (NH₄)₂SO₄, 4mM DTT, 200 μM (dATP, dGTP, dCTP, and dTTP each), 100 μM CaCl₂, and 120 nM Phi29 DNA polymerase. The reaction was initiated with the addition of 10 mM MgCl₂ and 1 μM of a single stranded oligonucleotide trap. The fluorescence of the reaction was read on a Molecular Dynamics M5 plate reader, again looking at fluorescence signal resulting from displacement of the quencher labeled complement oligonucleotide. As shown in FIG. 4, all three constructs of the 9° North polymerase, with and without polymerase activity, were able to prevent replication of the underlying template by the highly processive phi29 polymerase.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

What is claimed is:

1. A method of controlling a reaction with a nucleic acid, comprising:
providing the nucleic acid comprising at least one uracil containing nucleotide;
providing a nucleic acid binding protein with specificity for binding uracil containing nucleic acids in contact with the nucleic acid, under conditions whereby the nucleic acid binding protein binds to a portion of the nucleic acid comprising the uracil containing nucleotide, whereby binding of the nucleic acid protein to the nucleic acid at least partially blocks the reaction with the nucleic acid.

2. The method of claim 1, wherein the reaction comprises a ligation reaction, a polymerization reaction, an exonuclease reaction, an endonuclease reaction, a protection reaction, and/or a hybridization reaction.

3. The method of claim 2, wherein the reaction comprises a polymerization reaction.

4. The method of claim 1, wherein the nucleic acid binding protein comprises an archeal polymerase, a uracil-DNA glycosylase, a uracil binding fragment or a construct thereof.

5. The method of claim 4, wherein the nucleic acid binding protein comprises an archeal polymerase, a uracil binding fragment or a construct thereof.

6. The method of claim 2, wherein the reaction comprises a hybridization reaction and a reagent in the reaction comprises a polynucleotide complementary to at least a portion of the nucleic acid bound by the nucleic acid binding protein.

7. The method of claim 2, wherein the reaction comprises a protection reaction, and a reagent in the reaction comprises an exonuclease or an endonuclease, and the nucleic acid binding protein reduces activity of such endonuclease or exonuclease on at least a portion of the nucleic acid bound by the nucleic acid binding protein.

8. A composition, comprising:
a nucleic acid comprising one or more uracil containing bases;
a nucleic acid binding protein with specificity for binding uracil containing nucleic acids, bound to the nucleic acid; and
a reagent capable of reacting with the nucleic acid, wherein presence of a bound nucleic acid binding protein at least partially blocks the reagent from reacting with the nucleic acid.

9. The composition of claim 8, wherein the reagent comprises an enzyme selected from the group consisting of a polymerase, a ligase, a transcriptase, an endonuclease and an exonuclease.

10. The composition of claim 9, wherein the reagent comprises a polymerase.

11. The composition of claim 8, wherein the nucleic acid binding protein comprises an archeal polymerase, a uracil-DNA-glycosylase, a uracil binding fragment or a construct thereof.

12. The composition of claim 11, wherein the nucleic acid binding protein comprises an archeal polymerase, a uracil binding fragment or a construct thereof.

13. The composition of claim 12, wherein the archeal polymerase comprises 9° North polymerase, a uracil binding fragment or a construct thereof.

14. The composition of claim 13, wherein the 9° North polymerase comprises a polymerase inactive construct of 9° North polymerase.

15. The composition of claim 13, wherein the 9° North polymerase comprises an exonuclease deficient 9° North polymerase.


SEQUENCE LISTING (4 sequences)

SEQ ID NO: 1 - protein, 6 aa, Artificial Sequence
  Description of Artificial Sequence Synthetic peptide
GVLLLN

SEQ ID NO: 2 - DNA, 52 bp, Artificial Sequence
  Description of Artificial Sequence Synthetic oligonucleotide
tcgagcacgcggcacttattgcaauagtgccgagtcagcgcgctgactcggc

SEQ ID NO: 3 - DNA, 22 bp, Artificial Sequence
  Description of Artificial Sequence Synthetic oligonucleotide
gcaataagtgccgcgtgctcga

SEQ ID NO: 4 - DNA, 70 bp, Artificial Sequence
  Description of Artificial Sequence Synthetic oligonucleotide
tcgagcacgcggcacttattgcaauaggaaattaccctttatgcgtgccgagtcagcgcgctgactcggc